(19) United States
(12) Patent Application Publication
(10) Pub. No.: US 2022/0245836 A1
(43) Pub. Date: Aug. 4, 2022

(54) SYSTEM AND METHOD FOR PROVIDING MOVEMENT BASED INSTRUCTION

(71) Applicant: Altis Movement Technologies, Inc., Boca Raton, FL (US)

(72) Inventors: Jeff Halevy, Fort Lauderdale, FL (US); Constantine Goltsev, Kyiv (UA); Oleksii A. Bulgakov, Staten Island, NY (US); Aleksandr V. Tselousov, Kovrov (RU); Fedor G. Savchenko, Krasnador (RU)

(73) Assignee: Altis Movement Technologies, Inc., Boca Raton, FL (US)

(21) Appl. No.: 17/538,631

(22) Filed: Nov. 30, 2021

Related U.S. Application Data
(60) Provisional application No. 63/168,790, filed on Mar. 31, 2021.
(60) Provisional application No. 63/145,244, filed on Feb. 3, 2021.

(51) Int. Cl. G06T 7/246; G06V 40/20; G06T 19/20; G06T 19/00; A63B 71/06; A63B 24/00

(52) U.S. Cl. CPC G06T 7/251; G06V 40/23; G06T 19/20; G16H 20/30; A63B 71/0622; A63B 24/0062; G06T 19/006

(57) ABSTRACT

A method, comprising generating, for display to a user, a representation of the user performing a movement pattern of an activity from a first point of view; sensing a deviation of movement of the user from a model movement pattern for the activity; selecting a second point of view based on a type of the deviation; and generating, for display to the user, a representation of the user performing the movement pattern for the activity from the second point of view.

### RELATED APPLICATIONS

This application claims the benefit of priority to U.S. Provisional Patent Application Ser. No. 63/145,244, filed Feb. 3, 2021 and titled “SYSTEM AND METHOD FOR GENERATING MOVEMENT BASED INSTRUCTION” and U.S. Provisional Patent Application Ser. No. 63/168,790, filed Mar. 31, 2021 and titled “SYSTEM AND METHOD FOR GENERATING MOVEMENT BASED INSTRUCTION”. The disclosures of these prior Applications are considered part of and are incorporated by reference in the disclosure of this Application.

### BACKGROUND

Motion capture devices comprise image sensors that capture positional data within the view of the image sensors. Image data is processed to provide novel systems and methods for movement based instruction as described herein.

### BRIEF DESCRIPTION OF THE DRAWINGS

FIG. 1 illustrates a system to provide movement based instruction to a user in accordance with certain embodiments.

FIG. 2A illustrates a display of a representation of a user in accordance with certain embodiments.

FIG. 2B illustrates a representation of a user from multiple points of view in accordance with certain embodiments.

FIG. 3 illustrates a display comprising a representation of a user, a representation of a trainer, and a repetition tracker in accordance with certain embodiments.

FIG. 4 illustrates a display comprising a representation of a user, a most recent score, and a score history in accordance with certain embodiments.

FIG. 5 illustrates a series of images that may be generated by system and displayed to provide movement instruction to user.

FIG. 6 illustrates an example series of images that may be generated by system and displayed to provide movement instruction to user.

FIG. 7 illustrates a flow for providing movement based instruction in accordance with certain embodiments.

FIGS. 8A-8D illustrate example configurations utilizing various motion capture techniques in accordance with certain embodiments.

FIGS. 9A-9D illustrate various views of a computing device incorporating various components of a motion capture and feedback system in accordance with certain embodiments.

FIGS. 10A-10B illustrate various views of another computing device incorporating various components of a motion capture and feedback system in accordance with certain embodiments.

FIG. 11 illustrates example segments of a body in accordance with certain embodiments.

### DETAILED DESCRIPTION

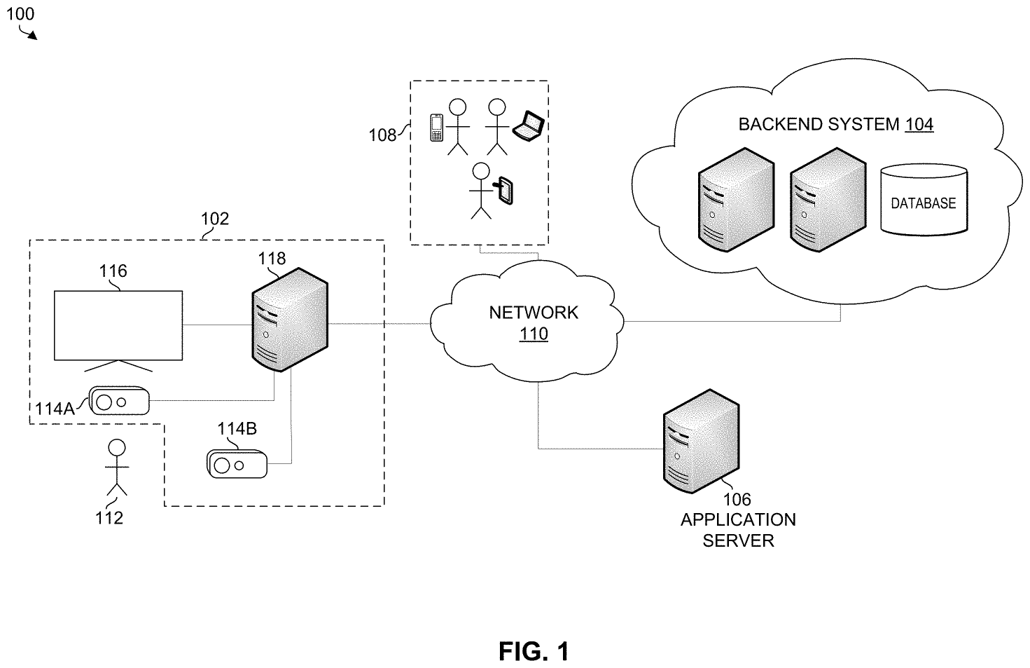

FIG. 1 illustrates a system 100 to provide movement based instruction to a user 112 in accordance with certain embodiments. System 100 includes a motion capture and feedback system 102, backend system 104, application server 106, and expert network system 108 coupled together through network 110. The system 102 includes motion capture devices 114 (e.g., 114A and 114B), display 116, and computing device 118. Other embodiments of system 100 may include other suitable components or may omit one or more of the depicted components.

Many individuals find fulfillment and/or enjoyment in movement based activities such as physical fitness exercises like calisthenics, plyometrics, weightlifting, and the like, as well as sports activities (such as pitching or hitting a baseball, dribbling or shooting a basketball, swinging a golf club, and the like), dance, and the like, as a means to reduce stress, increase muscle mass, improve bone strength, increase overall fitness, and to otherwise enhance quality of life. However, learning new movements and correctly performing these movements may be difficult, particularly for a beginner. In many instances, movement based activities may be difficult to optimally or safely perform without specialized training. A typical way to obtain this specialized training is through an in-person lesson with a personal trainer, coach, or other instructor. However, such training is subject to availability of a suitable instructor and may be cost prohibitive, depending on the trainee's situation and the duration of the training. Moreover, the quality of such instruction is highly variable as it is dependent on the ability, knowledge, and temperament of the instructor. Indeed, finding a properly qualified and reasonably priced instructor who has a schedule that aligns with an individual may be difficult or impracticable in many situations. One could alternatively seek to self-train by learning about a movement through print or video instruction and then attempting to implement the instruction. However, it may be difficult, time consuming, and/or potentially dangerous to learn and improve movements in this manner without real time feedback on proper performance of the movement.

In various embodiments of the present disclosure, system 100 may function as a computer-based, or artificially intelligent, personal trainer. System 100 may provide general instruction regarding movement based activities and may utilize motion capture devices 114 to record the movement of a user 112 performing an activity in order to provide personalized feedback to the user in real time. In various embodiments, the system 100 may provide information to correct the movement form of the user to promote health, safety, and optimal results. In some embodiments, the movement of a user may be mapped into a three-dimensional space and compared to a model movement form in the three-dimensional space in order to generate personalized instruction for the user 112. In various embodiments, system 100 may utilize multiple motion capture devices 114 in order to enable display of the user from a point of view that is adapted to the particular corrective instruction (e.g., based on the movement errors committed), enabling the user to quickly visualize and improve movement form. Thus, various embodiments may provide one or more advantages over other methods of movement instruction, such as improved instruction quality utilizing artificial intelligence (AI) techniques to provide hyper-personalized expert instruction, on-demand training, cost effective instruction, or real-time feedback.

Various embodiments of the present disclosure include a system 100 providing an intelligent personal trainer to provide instruction and real-time feedback for movement activities. The system 100 includes a motion capture and feedback system 102 operable to track the motion of a user 112 performing a movement activity, analyze the motion with respect to model movement patterns, and provide real-time feedback and encouragement to the user 112 to promote healthy and optimal movement patterns.

In operation, the system 100 may display (e.g., via display 116) a demonstration of an example movement pattern (e.g., a video or other visual representation) for a movement activity to be performed by the user 112. After viewing the demonstration (or independent of the demonstration), the user 112 may perform one or more repetitions of the movement activity. The system 100 may utilize a mapping of a movement activity in a three dimensional (3D) space and compare it against the movement pattern of the user during these repetitions. The system 100 may then provide real-time feedback to the user, including confirmation that the movement activity was performed correctly or specific instruction as to how to improve the movement pattern. When providing feedback, the system 100 may display (e.g., via display 116) the feedback from an optimal point of view that is selected by the system 100 based on the feedback being provided, allowing the user to clearly discern the portion of the user's movement that should be improved. In various embodiments, the system 100 may be capable of displaying any arbitrary point of view around the user as the optimal view to provide feedback, thus the user need not rotate his or her body in order to see a portion of the body that is the subject of the feedback (as would be required if a user were looking at a mirror for visual feedback). For example, the user may maintain a single orientation, while different feedback provided by the system 100 may display the user from the front, side, back, or other suitable point of view with accompanying corrective feedback.

In this manner, system 100 may function as an on-demand expert personal trainer, teaching the user 112 new movement activities and aiding the user in performing movement activities in a safe and effective manner, while lowering the cost and increasing the convenience of a workout session with expert instruction. The system 100 may provide the personal training functionality described herein for any number of users 112. For example, the system 100 may be used privately in a home gym, publicly in a commercial gym, or in any other suitable setting.

While this disclosure will focus on application of the system 102 to movement activities such as weightlifting exercises, the system 100 may be configured to provide instruction for any suitable movement activities, such as plyometrics, dancing, running, playing musical instruments, or sport-specific athletic movements such as pitching or hitting a baseball, dribbling or shooting a basketball, throwing a football, swinging a golf club, spiking a volleyball, and the like.

In system 100 of FIG. 1, motion capture devices 114A and 114B may capture multiple images (e.g., 2D or 3D images) of the user 112 over a time period to produce a video stream (e.g., a temporally ordered sequence of 2D or 3D images). In order to capture the images, a motion capture device 114 may include one or more image sensors, e.g., light detection and ranging (LIDAR) sensors, two-dimensional (2D) cameras (e.g., RGB cameras), ultrasonic sensors, radars, or three-dimensional (3D) or stereo cameras (e.g., depth sensors, infrared illuminated stereo cameras, etc.).

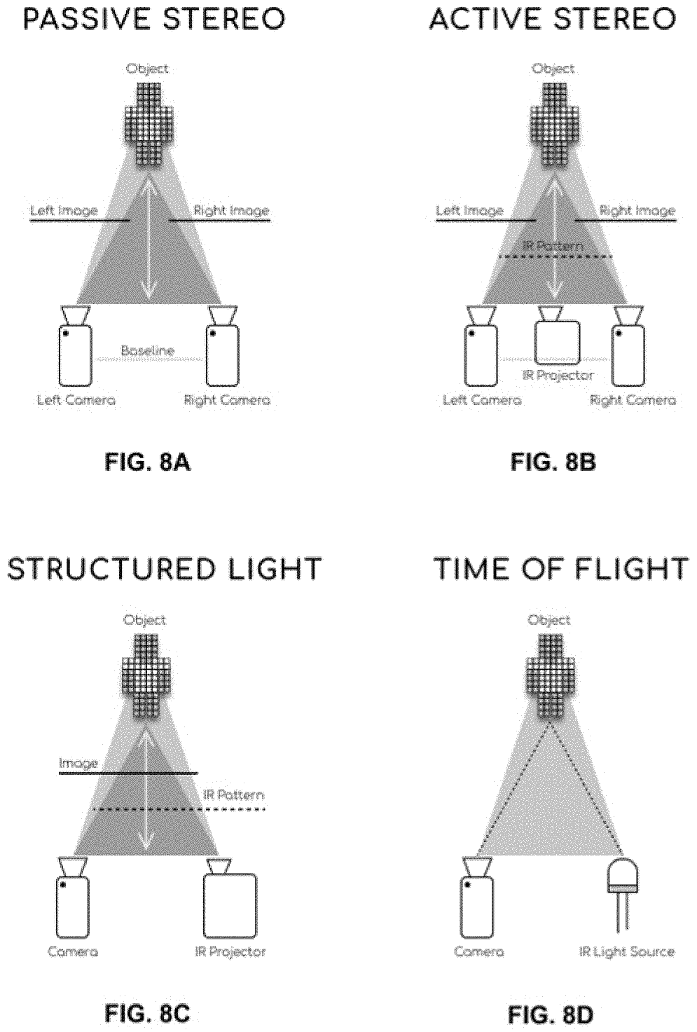

In various embodiments, the motion capture devices 114 of system 100 may utilize one or more of passive stereo, active stereo, structured light, or time of flight image acquisition techniques (if more than one technique is used, the acquired images may be fused together). FIGS. 8A-8D illustrate example configurations utilizing such techniques. FIG. 8A illustrates a passive stereo configuration and FIG. 8B illustrates an active stereo configuration. In both configurations, two cameras (depicted as a right camera and a left camera) capture slightly different images which may be used to generate a depth map. In a passive stereo configuration, an active light source is not used, while in an active stereo configuration, an active light source (e.g., a projector) is employed. FIG. 8C illustrates a structured light configuration in which a modulated light pattern is transmitted (e.g., by a projector) to the surface of a scene and an observed light pattern deformed by the surface of the scene is compared with the transmitted pattern and the image is obtained based on the disparity determined by the comparison. Although a single camera is depicted in FIG. 8C, in other structured light configurations, multiple (e.g., at least two) cameras may be used. FIG. 8D illustrates a time of flight configuration. In this configuration, the distance between the camera and an object is calculated by measuring the time it takes a projected light to travel from the infrared light source emitter, bounce off the object surface, and return to the camera receiver (based on the phase shift of the emitted and returned light). The object may then be reconstructed in an image based on such measurements.

Returning to FIG. 1, in various embodiments, a motion capture device 114 may include more than one image sensor. For example, a motion capture device 114 comprising a stereo camera may include two RGB cameras to capture 2D images. As another example, a motion capture device 114 may comprise two calibrated RGB cameras with a random infrared pattern illuminator. As another example, a motion capture device 114 may include a depth sensor as well as an RGB camera. In various embodiments, when multiple motion capture devices 114A and 114B are used, the sensors of the motion capture devices may be of the same type (e.g., the same one or more image sensors are resident on each motion capture device 114) or of different types (e.g., 114A may include an RGB camera and 114B may include a LIDAR sensor).

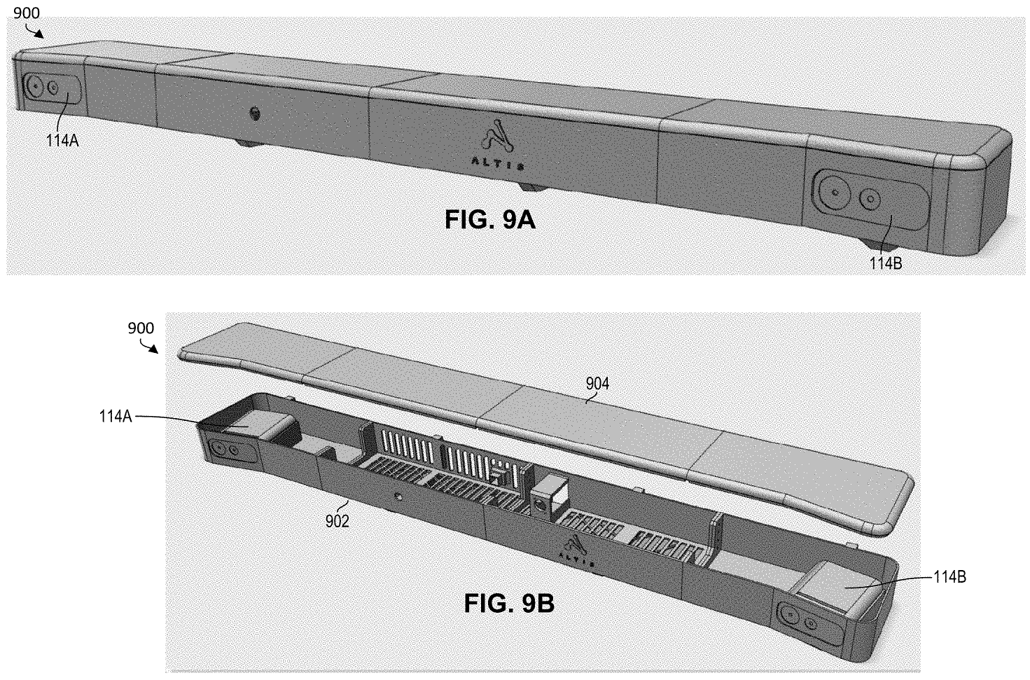

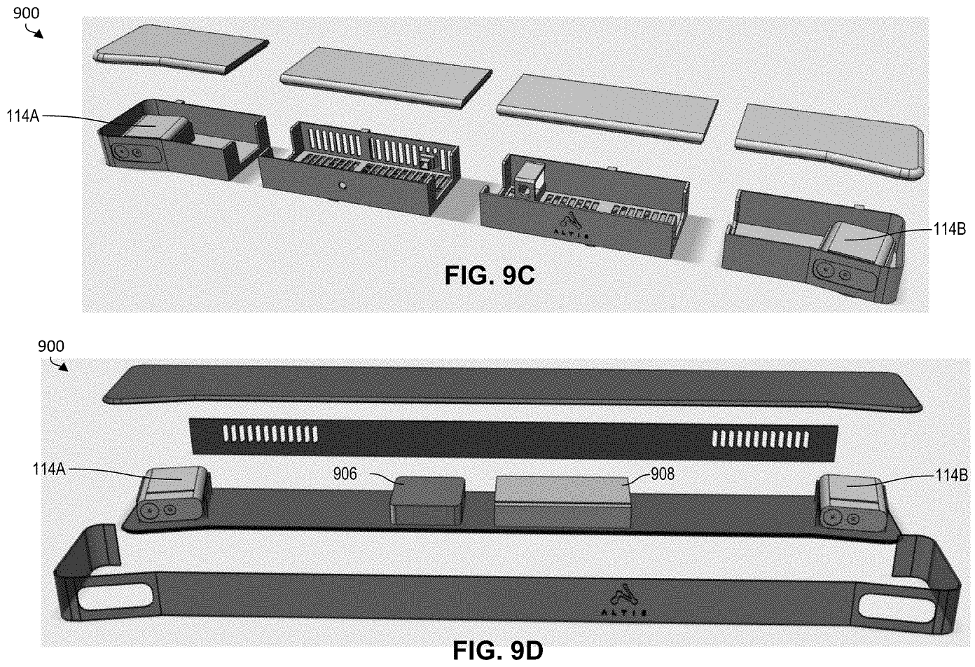

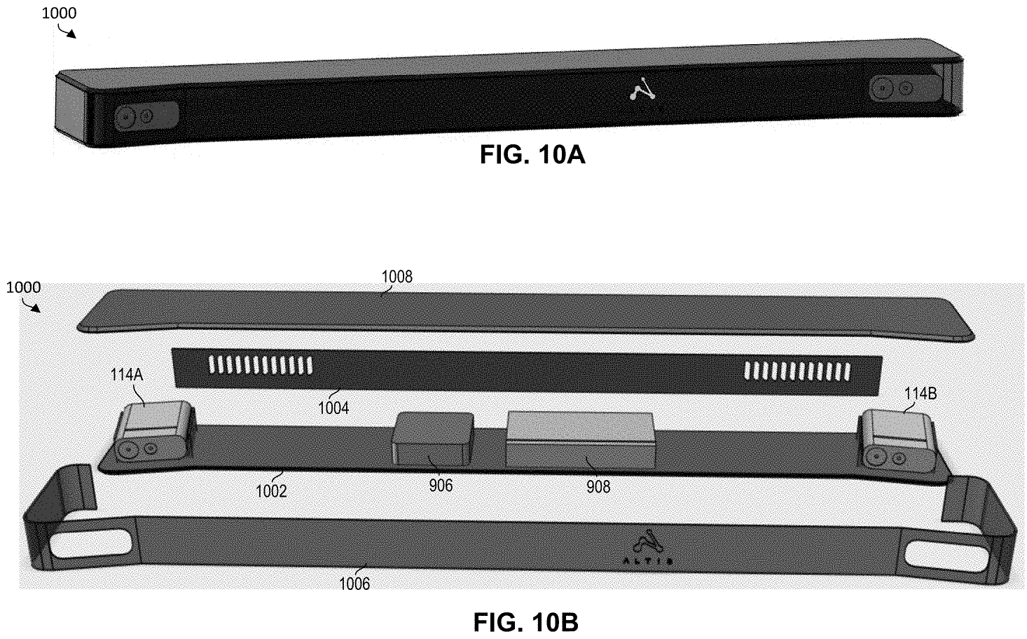

In the embodiment depicted, two discrete motion capture devices 114 (114A and 114B) are shown as being located in different positions so as to capture the user 112 at multiple different angles (whereas multiple image sensors on the same motion capture device 114 would capture the subject from substantially the same angle unless the motion capture device 114 is relatively large). In other embodiments, one or more additional motion capture devices 114 may be employed. In general, motion capture and feedback system 102 includes any suitable number and types of motion capture devices placed at different poses relative to the user 112 to enable capture of sufficient data to allow position determination of a group of body parts (which in some embodiments may be arranged into a skeleton) of the user 112 in 3D space, where a pose refers to the position and orientation of a motion capture device with respect to a reference coordinate system. For example, in the embodiment depicted, a motion capture device 114A is placed directly in front of the user 112 and a second motion capture device 114B is placed to the side of the user 112 (such that the angle formed between the first device, the user 112, and the second device is roughly 90 degrees in a horizontal plane). As another example, two motion capture devices may be placed at least a threshold distance apart (e.g., 5 feet) and may each be oriented towards the subject (e.g., one at a 45 degree angle and one at a −45 degree angle in a horizontal plane with respect to the user 112). As another example, two motion capture devices may be placed about 50 inches apart and each motion capture device may be angled inwards (e.g., towards the subject) by roughly 10 degrees. As another example, four motion capture devices may be placed as vertexes of a square and may be oriented towards the center of the square (e.g., where the user 112 is located). In various embodiments, the motion capture devices 114 may be placed at the same height or at different heights. FIGS. 9A-9D and 10 (to be discussed in further detail below) illustrate example configurations of motion capture devices 114. In some embodiments, cameras may be placed at the same horizontal position while each camera has its own vertical inclination. In some embodiments, a mechanism such as a scissor lift mechanism may be used to incline an apparatus containing the sensors.

In various embodiments, each motion capture device 114 may be discrete from each other motion capture device 114. For example, a motion capture device 114 may have its own power supply or its own connection (e.g., wired or wireless) to the computing device 118 to send data captured by its image sensor(s) (or data processed therefrom) to the computing device 118 (or other computing device performing operations for the system 102).

In some embodiments, the user 112 may wear special clothing or other wearable devices and locations of these wearable devices may be tracked by system 102 in order to capture the position of various segments (e.g., body parts) of user 112. In some embodiments, the wearable devices may be used to estimate the 3D positions of various segments of user 112 to supplement data captured by one or more motion capture devices 114 in order to improve the accuracy of the position estimation. In yet other embodiments, the wearable devices may be used to estimate the 3D positions of the segments of user 112 without the use of passive sensors such as cameras.

Motion capture and feedback system 102 (either by itself or in conjunction with one or more other devices of system 100) may track the movement of the user 112 by obtaining data from motion capture devices 114 and/or wearable devices and transforming or translating the captured data into representations of three dimensional (3D) positions of one or more segments (e.g., body parts) of the user 112. As examples, such segments may include one or more of a head, right and left clavicles, right and left shoulders, neck, right and left forearms, right and left hands, chest, middle spine, lower spine, right and left thighs, hip, right and left knees, and right and left feet.

Data captured by motion capture devices 114 may be processed by the system 102 (e.g., via computing device 118 or processing logic of one or more motion capture devices 114) and/or other system (e.g., backend system 104) to form a 3D model of the user's position as a function of time. Such processing may utilize any suitable collection of information captured by system 102, such as 2D images, 3D images, distance information, position information, or other suitable information.

In one embodiment, system 102 captures 3D point clouds that may be input into a neural network (e.g., that executes an artificial intelligence (AI) function) or other logic to reconstruct the user's body segments (e.g., in the form of a skeleton) in 3D space. In another embodiment, system 102 uses two or more motion capture devices 114 each comprising at least one RGB sensor and provides captured data to a neural network or other logic to construct a 3D skeleton directly. The neural network or other logic used to determine the user's position in 3D space may be implemented in whole or in part by computing device 118, one or more motion capture devices 114 (e.g., by processing logic resident thereon), or other system (e.g., backend system 104). In one embodiment, the computing device 118 may communicate captured data (e.g., raw image data and/or processed image data) to one or more other computing devices (e.g., within backend system 104) for processing. Various embodiments may employ different types of processing locally (e.g., by computing device 118) or remotely (e.g., by backend system 104). For example, in one embodiment, the computing device 118 may compress the raw image data and send it to a remote system for further processing. As another example, the computing device 118 may locally utilize a neural network to execute an AI function that identifies the user's segments (e.g., skeleton) without involving a remote system for the segment detection.

In various embodiments, display 116 (which may, e.g., comprise any suitable electronic display) may provide instruction associated with movement of the user 112. In various embodiments, the determination and/or generation of the instruction to provide to the user via display 116 may be performed by computing device 118, backend system 104, or a combination of computing device 118 and backend system 104. In some instances, the user 112 may be oriented in a forward position (e.g., facing the display 116) during an exercise so that the user can view instruction provided via the display 116. In some embodiments, display 116 may be integrated with computing device 118 or coupled thereto.

A user 112 may issue commands to control the system 102 using any suitable interface. In a first example, user 112 may issue commands via body movements. For example, the user 112 may raise an arm to initiate a control session and then move the arm to select a menu item, button, or other interface element shown on the display 116. The display may update responsive to movement of the user 112. For example, a cursor may be displayed by the display 116 and movement of the user may cause the cursor to move. When a user's hand position (e.g., as indicated by the cursor) corresponds with an interface element on the display 116, the interface element may be enlarged or highlighted and then the user may perform a gesture (e.g., make a first or wave a hand) to cause the system 102 to initiate the action that corresponds to the interface element. Thus, in one example, the user 112 may control the system 102 using contactless gestures. As another example, the system 102 may comprise a directional microphone (e.g., the microphone may be integrated with computing system 118 and/or display 116) that accepts voice commands from the user 112 to control the system 102. In one such example, the user 112 may initiate control by saying a key word which prompts the system 102 to listen for a voice command. As yet another example, a user 112 may control the system 102 by using an application on a mobile or other computing device that is communicatively coupled (e.g., by network 110 or a dedicated connection such as a Bluetooth connection) to the computing system 118. In this example, the device may be used to control the system 102 (e.g., navigate through an interface, enter profile information, etc.) as well as receive feedback from the system (e.g., workout statistics, profile information, etc.). In various embodiments, system 102 may implement any one or more of the above examples (or other suitable input interfaces) to accept control inputs.

In various embodiments, system 102 renders the dynamic posture of the user 112 on the display 116 in real-time during performance of an activity by the user. Thus, when the user views the display 116, the user may monitor his or her movement as if the display 116 were a mirror.

In various embodiments, display 116 may display a trainer performing an example movement pattern for an activity to the user 112. The example movement pattern may take any suitable form (such as any of the representation formats described below with respect to a trainer or user 112). In some embodiments, the display of the trainer may be video (or a derivation thereof) of one or more experts of expert network system 108 (or another user 112 of the system 100 that is deemed to have acceptable form) performing the movement.

In various embodiments, the trainer may be displayed simultaneously with the user 112 or the system 102 may alternate between display of the trainer and the user. The trainer may be displayed at any suitable time, such as before the user 112 performs a repetition of the activity, responsive to a request from the user 112, and/or responsive to a movement error by the user 112 performing the activity.

Any suitable representation of the user 112 or trainer may be displayed. For example, display 116 may display a visual representation of a 3D positional data set of a user 112 or trainer performing an activity, where a 3D positional data set may include any suitable set of data recorded over a time period allowing for the determination of positions of segments of a user 112 or trainer in a 3D space as a function of time. For example, a 3D positional data set may include a series of point clouds. As another example, a 3D positional data set may include multiple sets of 2D images that may be used to reconstruct 3D positions. As yet another example, a 3D positional data set may include a set of 2D images as well as additional data (e.g., distance information). The visual representation may include a video or an animation of the user 112 or trainer based on a respective set of 3D positional data (e.g., point clouds).

In some embodiments, when a 3D positional data set is displayed, a representation of the user 112 or trainer may be displayed along with detected parts of the body of the user 112 or trainer. For example, particular joints and/or body segments of the user 112 or trainer may be displayed. In some embodiments, a skeleton may be constructed from the detected body parts and may be displayed. In various embodiments, the processing to detect body parts from the raw image and/or positional data may be done in whole or in part by the computing device 118 or may be performed elsewhere in system 100 (e.g., by backend system 104, and/or one or more motion capture devices 114).

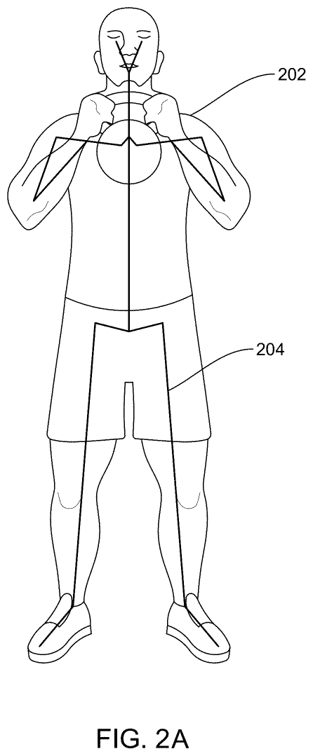

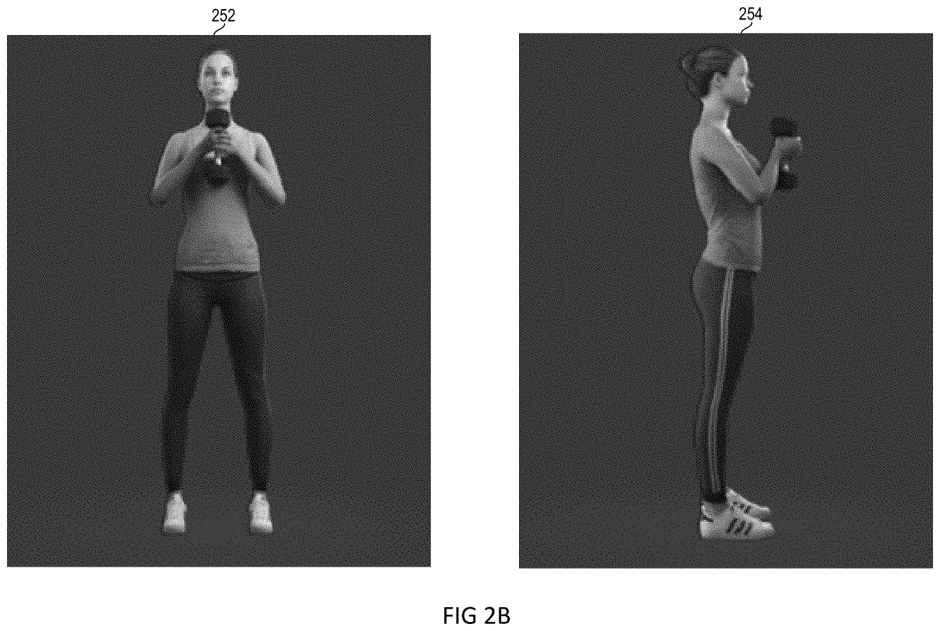

FIG. 2A illustrates a display of a representation 202 of a user 112 in accordance with certain embodiments. In the embodiment depicted, the representation 202 of the user 112 as well as a skeleton 204 of detected body parts (e.g., joints or other body segments) along with connections between the body parts of the user 112 is displayed (where the skeleton may be overlaid on the representation 202 of the user 112). In some embodiments, the skeleton 204 is displayed in the same 3D space along with the representation 202. In other embodiments, the skeleton of the subject 112 may be displayed separately from the representation 202 or the representation 202 of the subject 112 may be omitted altogether and only the skeleton 204 displayed in some embodiments (and thus the skeleton itself could be the representation of the user 112 that is displayed).

In some embodiments, the representation 202 may comprise a series (in time) of colored images of the user 112. In one embodiment, the representation 202 may include only the joints of the subject. In another embodiment, the representation 202 may include the joints as well as additional visual data, such as connections between the joints. In various embodiments, a representation 202 may include a view of the entire user 112 as captured by the motion capture devices 114 and transformed (e.g., via a matrix) to the desired orientation, a view of a skeleton or other key points of the user 112, an avatar of the user 112 or superimposed on a representation of the user 112 (e.g., the representation 202 may be a simulated human or avatar with movements governed by the 3D positional data set), or an extrapolation of the images captured by motion capture devices 114 (e.g., a view of the user's back may be extrapolated from the captured data even when respective motion capture devices do not capture an image of the user's back directly). As described above, the form of any of the example representations of the user 112 may also be used as the form of representation of the trainer when the trainer is displayed.

FIG. 2B illustrates a representation of a user 112 from multiple points of view in accordance with certain embodiments. When the user 112 is performing an activity, the system may display the user from a default point of view as depicted in representation 252. Responsive to a determination that the movement of the user 112 is suboptimal, the system may change the point of view of the displayed representation based on the type of mistake made by the user. Representation 254 shows the user from a different point of view. The point of view in representation 254 may be displayed by the system, e.g., until the user 112 corrects the mistake or the system otherwise determines that a different point of view should be shown. More detail on how the system may determine which point of view to display is provided below.

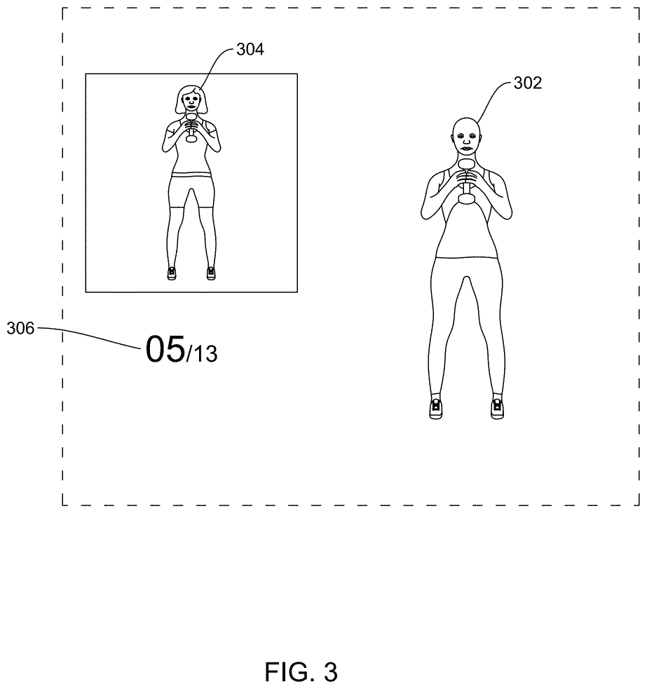

FIG. 3 depicts a display comprising a representation 302 of a user 112, a representation 304 of a trainer, and a repetition tracker 306 in accordance with certain embodiments. In the depicted embodiment, system 102 may display, via repetition tracker 306, a number of repetitions of an activity that have been performed by the user 112. In some embodiments, the repetition tracker 306 may also display the number of target repetitions to be performed by the user 112 (and when the number of target repetitions is reached, the system 102 may transition, e.g., to the next activity or next set of the same activity).

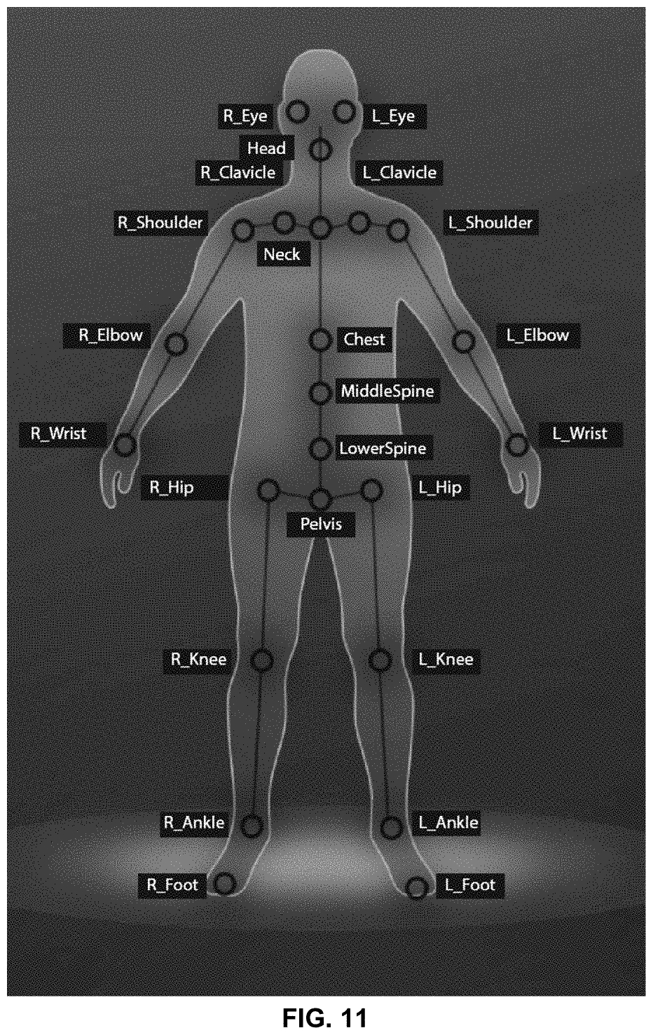

In various embodiments, in order to enable counting of repetitions, an activity may be associated with one or more phases. A phase may be associated with one or more segments (e.g., body parts such as a joint or other portion of the subject 112) and corresponding positions of the one or more segments in a 3D space. For example, as illustrated in FIG. 11, such segments may include one or more of a head, right and left eyes, right and left clavicles, right and left shoulders, neck, right and left elbows, right and left wrists, chest, middle spine, lower spine, right and left hips, pelvis, right and left knees, right and left ankles, and right and left feet. Other embodiments may include additional, fewer, or other body parts that may be associated with a phase. The illustrated segments (or variations thereof) may similarly be used for any of the skeletons (e.g., detected skeleton, guide skeleton, etc.) described herein.

A 3D position associated with a segment for a phase may be represented as an absolute position in a coordinate system, as a relative position within a range of positions (so that the data may be used for subjects or users of various shapes and sizes), or in other suitable manner. These position(s) may be used for comparison with corresponding positions of 3D positional data of a user 112 to determine how closely the body positions of the users 112 match the stored body positions of the phases in order to determine when a phase has been reached during movement of the user 112.

As one example, for an activity such as a squat type, the activity may have a top phase and a bottom phase. When the system detects that the user 112 has reached the bottom phase and then the top phase, the system 102 may increment the counter. Thus, the configured phases may be utilized by the system 100 to implement a counter that tracks the number of repetitions of the activity that have been performed. In some embodiments, an activity may include a single phase or more than two phases.

In some embodiments, the phases set for an activity may additionally or alternatively be used to determine how closely the form of the user 112 matches a model movement form (in other embodiments the form of the user 112 may be compared with the model movement form without using such phases).

The comparison between the movement of the user 112 and the model movement form may be performed using any suitable collection of data points representing one or more positions of body parts of a user 112. For example, joints, segments coupled to one or more joints, and/or angles between segments of a detected skeleton of the user may be compared with corresponding joints, segments, and/or angles in a piecewise fashion (or a combination of certain joints, segments, or angles may be compared against corresponding combinations) of a model movement pattern.

In various embodiments, the difference between the user's movement and the model movement pattern may be quantified using any suitable techniques (e.g., linear algebra techniques, affine transformation techniques, etc.) to determine the distances between the model 3D positions of the selected body parts (e.g., as defined by the phases of the activity or otherwise defined) versus the detected 3D positions during a repetition performed by user 112. In various embodiments, the difference may be determined based at least in part on Euclidean distances and/or Manhattan distances between model 3D positions and detected 3D positions. In some embodiments, a relative marker such as a vector from a detected body part towards the model 3D position may be used in conjunction with the distance between the detected body part and the model 3D position to determine a difference between the user's movement and the model movement pattern.

In various embodiments, the comparisons may be made for any number of discrete points in time over the course of the movement. For example, in some embodiments, the comparisons may be made for each defined phase of the activity. As another example, the comparisons may be made periodically (e.g., every 0.1 seconds, every 33.3 milliseconds, etc.) or at other suitable intervals. In some embodiments, the comparisons may involve comparing a value based on positions detected over multiple different time points (e.g., to determine a rate and/or direction of movement) with a corresponding value of a model movement pattern.

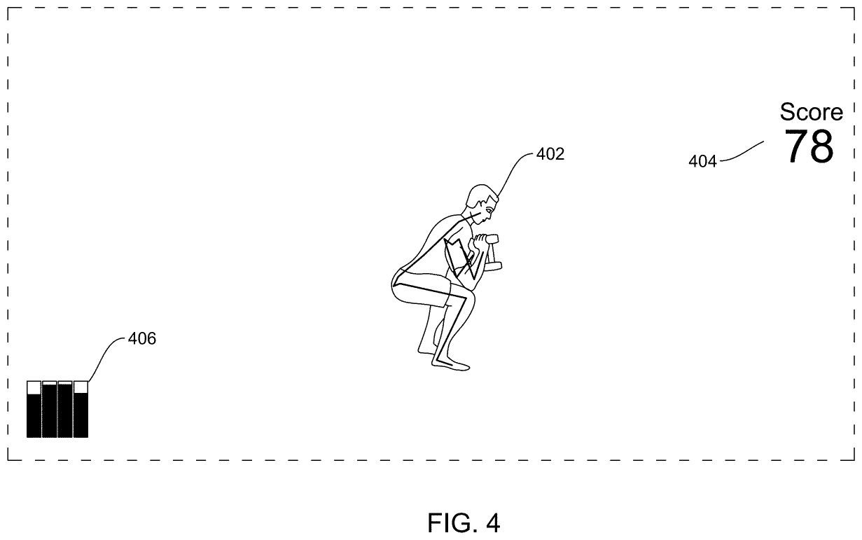

FIG. 4 depicts a display comprising a representation 402 of a user 112, a most recent score 404, and a score history 406 in accordance with certain embodiments. In various embodiments, system 102 may determine a score for a user's performance of a repetition of an activity. A score (e.g., 404) may indicate how closely the movement of the user 112 aligns with the model movement pattern which may be determined in any suitable manner, such as using any techniques described above. In the embodiment of FIG. 4, the score 404 of the latest repetition is shown in the upper right corner, while a bar graph representation of a score history 406 is shown in the lower left corner. The scores may provide instant feedback to a user 112 as well as allow a user 112 to see progress over time. In some embodiments, score histories from different activity sessions (e.g., performed on different days) are stored by the system 102 and the scores (or metrics based thereon, such as score averages) are made available to the user 112 (e.g., via display 116).

In various embodiments, system 102 may analyze movement form of the user 112 performing an activity and determine that the user has suboptimal movement form (also referred to herein as a “movement error”). In response, system 102 may alert the user 112 of the movement error. The determination that the user has suboptimal movement form may be made at any suitable granularity. For example, the determination may be made in response to the user committing a movement error during a repetition of an activity, a user committing a movement error for multiple consecutive repetitions of the activity, or a user committing a movement error for a certain percentage of repetitions of the activity.

The determination that the user has suboptimal movement form may be based on comparison of the movement of the user 112 with data representing a model movement pattern and/or data representing improper movement patterns (e.g., provided by expert network system 108). In various embodiments, deviations between the user's movement and a model movement pattern may indicate suboptimal movement form. For example, differences determined using any of the methods above (e.g., using comparisons between body parts of the user and the model movement form in a piecewise or aggregate fashion) that are above a certain threshold may indicate a suboptimal movement form. As another example, a similarity between the user's movement and an improper movement pattern may indicate a deviation from the model movement pattern and may thus indicate that a movement error has been committed. The determination that the user has suboptimal movement form may utilize any of the methods described above for comparing the user's form to the model movement pattern (and such methods may be adapted for comparing the user's form to one or more improper movement patterns).

A particular movement error may be associated with one or more body parts. When the position of these one or more body parts of the user 112 deviate from the position of the model movement pattern in a manner consistent with the movement error, the system 102 may detect that the user 112 has committed the movement error.

As an example, a movement error in which a user has a curved back during an activity (e.g., a squat) may be associated with the chest and the neck. As another example, a movement error in which a user has feet that are too narrow during the activity may be associated with the left and right feet. As yet another example, a movement error in which a user's knees cave outward during the activity may be associated with the left and right knee.

In some embodiments, the system 102 may also associate a weight with each body part associated with a movement error. The weight of a body part may indicate the relative importance of the body part in comparison of the user's movement form with a model movement pattern and/or one or more improper movement patterns. For example, if weights are used for a particular movement error and the chest is assigned a greater weight than the neck, then the position of the chest of the user 112 will be given greater relevance than the position of the neck in determining whether the user 112 has committed the movement error.

Different types of movement errors may have different thresholds for determining whether the movement error has been committed by the user 112, where one or more thresholds may be used in comparing the movement of the user 112 to the model movement pattern or one or more improper movement patterns. As various examples, a first movement error may be detected when a first body part deviates by more than a first threshold relative to a model movement pattern, a second movement error may be detected when a second body part deviates by more than a second threshold, a third movement error may be detected when a third body part deviates by more than a third threshold and a fourth body part deviates by more than a fourth threshold, and so on (similarly a threshold may be met when a user's body part deviates by less than the threshold from an improper movement pattern).

In some embodiments, system 102 may detect one or more of several types of movement errors associated with an activity. As just one example, a goblet squat activity may have detectable movement errors including “Not Utilizing the Full Squat”, “Feet too narrow”, “Rounded Back”, “Feet too wide”, “Knees Caving Outward”, and “Knees Caving Inward.” Each movement error could be associated with one or more different body parts, weights for the body parts, or comparison thresholds for determining whether the particular movement error has been committed by user 112. In some embodiments, each type of movement error may be associated with a distinct improper movement pattern that may be compared with the user's movement form.

Responsive to a determination that the user 112 has committed a movement error, the system 102 may provide instruction regarding how to improve the movement form. The instruction may be visual (e.g., displayed on display 116) and/or auditory (e.g., played through computing device 118 or display 116). In various embodiments, the system 102 may provide real time prompts to the user 112 to assist the user in achieving proper movement form. Alternatively or in addition, system 102 may store indications of prompts and provide the prompts at any suitable time. For example, the system 102 may provide prompts automatically when the corresponding movement errors are detected or provide the prompts responsive to a request from the user, e.g., after a workout set is completed, after an entire workout is completed, or prior to beginning a workout set (e.g., the prompts may be from a previous workout and the user 112 may desire to review the prompts for an activity prior to performing the activity again).

In some embodiments, the instruction provided may include a representation (e.g., an example movement pattern) of a trainer performing the activity (e.g., which may or may not be derived from a model movement pattern that is compared against the user's movement pattern). In various embodiments, responsive to a detection of a movement error, an example movement pattern of the trainer performing the activity is shown (e.g., from an optimal point of view) to illustrate how the movement error may be corrected. In some embodiments, the displayed example movement pattern of the trainer may include a full repetition of the activity or a portion of a repetition (e.g., to focus on the portion of the repetition in which the movement error was detected). In various embodiments, the specific movement and/or body parts associated with the movement error may be highlighted on the view of the trainer as the trainer moves through the particular activity.

In some embodiments, system 102 may provide an onscreen representation of the user 112 from an optimal point of view to highlight and correct the user's form. In one embodiment, when a user begins an exercise, the system 102 may display the user from a default point of view associated with the activity (different activities may have different default points of view). The system 102 may then change the point of view of the user 112 responsive to a determination that the user has committed a movement error (and that the optimal point of view is different from the default point of view). This may be performed without requiring the user 112 to change an orientation with respect to the motion capture devices 114 (e.g., the representation of the user 112 at the optimal point of view may be constructed from the data captured by motion capture devices 114).

In some embodiments, when a movement error is detected, the display of both the trainer and the user may be rotated to the same point of view associated with the particular movement error in order to illustrate the prescribed correction. The system may display the representation of the trainer or user in any suitable format (e.g., any of those described above with respect to representation 202 or in other suitable formats). Thus, in various embodiments, the system 102 may have the capability of rotating the user's image in 3D space to any suitable point of view and displaying an example movement pattern (e.g., of the trainer) alongside the user's actual movement at the same point of view (or a substantially similar point of view) in real time. In some embodiments, different points of view may be used for the representations of the trainer and for the user 112 for particular movement errors.

The particular point of view to be used to illustrate the correction of the error (e.g., by displaying the representation of the user 112 and/or the trainer) may be determined based on the type of movement error committed by the user 112. Each activity may be associated with any number of possible movement errors that are each associated with a respective optimal point of view of the user or trainer. Thus, when a movement error is detected, the associated optimal point of view is determined, and the representation of the user 112 or trainer is then displayed from that optimal point of view. For example, for a first type of movement error, the point of view may be a first point of view; for a second type of movement error, the point of view may be a second point of view; and so on. As just one example, if the movement error is an incorrect angle of the spine, the point of view may be a side view of the user or trainer, whereas if the movement error is an incorrect spacing of the feet, the point of view may be a front or back view of the user or trainer.

In some embodiments, a movement error may be associated with more than one optimal point of view. For example, the first time a movement error is detected, a first optimal view associated with the movement error is used to display the representation of the user or trainer while the second time (or some other subsequent time) the movement error is detected, a second optimal view associated with the movement error is used.

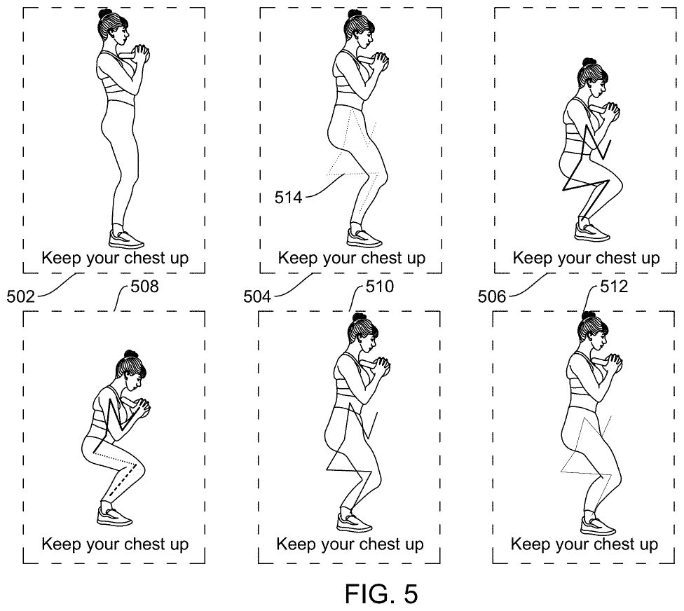

FIG. 5 illustrates a series of images that may be generated by system 102 and displayed (e.g., by display 116) to provide movement instruction to user 112. The images may be part of a video stream that is displayed by the display 116. In 502, the user 112 is beginning a repetition of an exercise. Because a suboptimal movement pattern has been detected (e.g., in a previous repetition of the exercise), the system 102 displays a corrective message: “Keep your chest up”.

In some embodiments, system 102 may display a guide skeleton 514 to preview the correct movement form. This guide skeleton 514 may be grounded at (e.g., anchored to) a base position equal to the user's current position (e.g., standing in the same spot as the user or otherwise aligned with the user), so that the user 112 does not need to change location to line up with the guide skeleton. In one embodiment, once the base position of the guide skeleton is established, the base position of the guide skeleton does not change for the remainder of an instance of the activity being performed (e.g., for a repetition or a set of repetitions of the activity).

In some embodiments, the guide skeleton may be selected based on the type of detected movement error as a fixed position depicted by the guide skeleton may be selected to illustrate a position that needs correction. As another example, the guide skeleton may be oriented from the optimal point of view associated with the movement error. In various embodiments, the guide skeleton is oriented from the same point of view as the representation of the user 112.

In some embodiments, the guide skeleton 514 will fade in gradually as a user gets close to a target position of the correction (e.g., as represented by a model position). For example, in 504, the user 112 begins squatting down and the guide skeleton 514 starts to fade in (illustrated by dotted lines). In 506, the user 112 is closer to the target position and the lines of the guide skeleton 514 are brighter than at 504.

In one embodiment, the guide skeleton 514 is a particular color (e.g., blue) by default and a portion or all of the guide skeleton may change color (e.g., to green) or brightness when the position of the detected skeleton of the user matches up with the guide skeleton. In one example, the guide skeleton may include multiple segments and each segment may individually change color when the corresponding segment of the user's detected skeleton matches up with the respective segment. In some embodiments, the color change is gradual and is based on a difference between the position of a segment of the guide skeleton and the corresponding segment of the user's detected skeleton. When the difference is larger, the color of the segment of the guide skeleton may include a larger component of the original color and as the difference decreases, the guide skeleton may include decreasing amounts of the original color (e.g., blue) and increasing amounts of the new color (e.g., green). When the difference is below a threshold (e.g., indicating that the position of that segment is correct), an additional color effect may be displayed (e.g., the segment may flash brightly or the skeleton segment may become thicker).

In 508, part of the user's body (e.g., the calf, fibula, and/or tibia) is aligned with the corresponding segment of the guide skeleton, another part (e.g., the femur or thigh) is almost aligned with the corresponding segment of the guide skeleton (and may be displayed differently, such as in a slightly different color and/or brightness which is represented by different dashing of the lines in 508), and the remaining portion of the user's body is not as closely aligned. The target position is not achieved by the user 112 in this illustration. At 510 and 512, the user returns towards an initial position and the guide skeleton fades away.

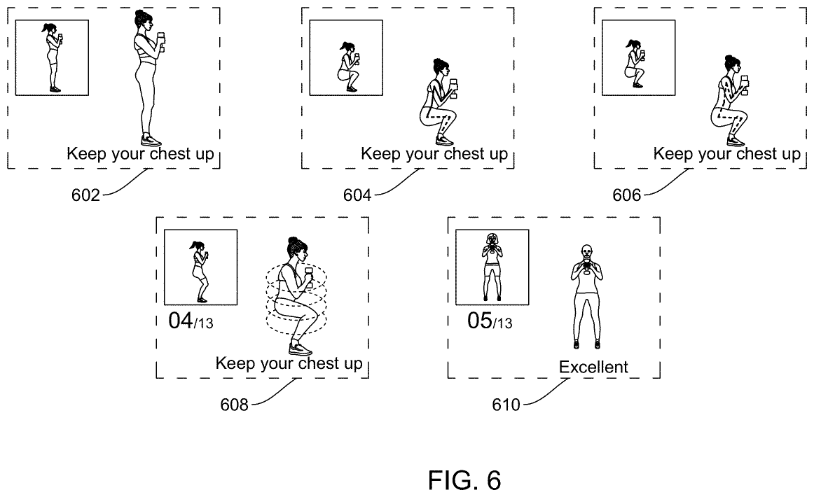

FIG. 6 illustrates an example series of images that may be generated by system 102 and displayed (e.g., by display 116) to provide movement instruction to user 112. The images may be part of a video stream that is displayed by the display 116. At 602, the user begins a repetition of an exercise. The display 116 shows the example movement pattern in the upper left corner and the representation of the user 112 in the middle. Because a suboptimal movement pattern has been detected (e.g., in a previous repetition of the activity), the point of view of the display of the user 112 (and the trainer) has been changed from the default point of view (which is shown at 610) to a side view to allow the user to view her chest in association with the personalized feedback (“Keep your chest up”) displayed by the system 102.

At 604, the user 112 has squatted down and is at or near the target position to be corrected. While the lower half of the user is correctly aligned with the guide skeleton, the upper half is still misaligned (in various embodiments, the misaligned segments may be a different color from the aligned segments, illustrated here by different dashing in the segments). At 606, the user has corrected position and the entire guide skeleton has turned the same color (illustrated by each segment having the same dashing). At 608 an animation is played wherein the guide skeleton disappears to indicate that the correct form has been attained and an encouraging message (e.g., “Excellent!”) is presented at 610.

In one embodiment, once all of the guide skeleton and user segments are successfully aligned (or it is otherwise determined that the movement error has been corrected), the point of view may transition back to the default view. For example, the point of view may be changed back to the initial point of view of the display of the user (e.g., a frontal point of view).

As depicted in FIGS. 5 and 6, the view showing the user 112 at the optimal point of view may also include one or more visual targets for the user's body parts so that the user can align with the proper form. The visual targets may include one or more of a written message with movement instruction such as “keep your chest up” or “bend your knees more”, an auditory message with movement instruction, or the guide skeleton showing a target position.

In some embodiments, system 102 may detect multiple errors in the user's movement over one or more repetitions. For example, during a repetition, system 102 may detect that a user's knees should bend more and the user's chest should be kept higher. In one embodiment, when multiple errors are detected, system 102 may focus its feedback on the most egregious error and utilize that error's associated optimal point of view and/or visual or audio prompt(s). Which movement error is most egregious could be determined in any suitable manner. For example, the movement error that is the most dangerous of the detected movement error could be selected as the most egregious movement error. As another example, the movement error that represents the furthest deviation from the model movement pattern may be selected as the most egregious movement error. In another example, the movement error that occurs earliest in a repetition may be corrected first, as subsequent movement errors may result from this movement error.

In some embodiments, instruction regarding one or more other detected errors may be provided at a later time (e.g., after the user has corrected the most egregious error). In other embodiments, system 102 may show correction for the multiple errors simultaneously or for multiple errors in succession. When correction is shown for multiple errors and the errors have different optimal viewpoints, the view could transition through optimal viewpoints associated with the movement errors (e.g., an optimal viewpoint associated with a first movement error may alternate with an optimal viewpoint associated with a second movement error). Alternatively, a viewpoint that is based on both optimal viewpoints may be used (e.g., a viewpoint that is in between the two optimal viewpoints that provides a balance between the two optimal viewpoints may be identified and used).

In various embodiments, system 102 may store activity profiles, where an activity profile includes configuration information that may be used to provide instruction for a specific activity, such as a weightlifting exercise (e.g., clean and jerk, snatch, bench press, squat, deadlift, pushup, etc.), a plyometric exercise (e.g., a box jump, a broad jump, a lunge jump, etc.), a movement specific to a sport (e.g., a baseball or golf swing, a discus throw), a dance move, a musical technique (e.g., a bowing technique for a violin, a strumming of a guitar, etc.), or other suitable movement pattern. An activity profile may be used by system 102 to provide feedback about the activity to any number of users 112.

For example, motion capture and feedback system 102 may track the motion of a user 112 performing an activity and compare positional data of the user 112 with parameters stored in the activity profile in order to provide feedback to the user 112 (e.g., by providing corrective prompts for mistakes and rotating a display of the user to an optimal point of view).

An activity profile for an activity may include one or more parameters used to provide instruction to a user 112. For example, the parameters of an activity profile may include any one or more of the following parameters specific to the activity (or any of the other information described above with respect to the features of the system 102): 3D positions for one or more specified segments of a subject (e.g., a trainer) at specified phases of a model movement pattern for an activity, 3D positions for one or more specified segments of a subject at specified phases for one or more movement errors, weights for the specified segments, parameters (e.g., thresholds) to be used in determining whether a mistake has been committed by a user 112, optimized points of view for correction of the one or more mistakes, and corrective prompts for the one or more mistakes.

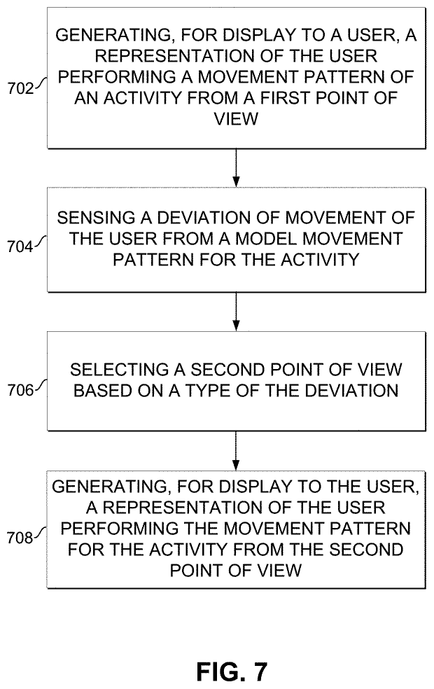

FIG. 7 illustrates a flow for providing movement based instruction in accordance with certain embodiments. At 702, a representation of the user performing a movement pattern of an activity from a first point of view is generated for display to a user. At 704, a deviation of movement of the user from a model movement pattern for the activity is sensed. At 706, a second point of view based on a type of the deviation is selected. At 708, a representation of the user performing the movement pattern for the activity from the second point of view is generated for display to the user.

FIGS. 9A-9D illustrates various views of a computing device 900 incorporating various components of system 100. For example, device 900 may include motion capture devices 114A and 114B as well as other components that may implement all or a portion of computing device 118. In the embodiments depicted, device 900 includes a housing comprising a housing base 902 and a housing lid 904 to be placed over the housing base. The housing encloses the components of the device 900. The housing base includes vents on the bottom and the rear for airflow and apertures for power and video cabling.

In some embodiments (e.g., as depicted in FIG. 9C), the housing base 902 and housing lid 904 may each comprise a plurality of sections that may be coupled together to form the housing.

Various computing components may be placed within the housing of device 900. In the embodiments depicted in FIGS. 9A-9D, motion capture devices 114A and 114B are placed proximate opposite ends of the housing and are angled slightly inwards (e.g., roughly 10 degrees) relative to the length of the housing. In one embodiment, the motion capture devices 114A and 114B are placed roughly 5 feet apart. In one embodiment, motion capture devices 114 are Azure Kinect or Kinect 2 devices utilizing time of flight imaging techniques.

As depicted in FIG. 9D, additional computing components 906 and 908 may be placed within the housing. Components 906 and 908 may include any suitable circuitry to provide functionality of the device 900 (which may implement at least a portion of computing device 118). For example, component 906 may be a power supply and component 908 may include a computing system comprising one or more of a processor core, graphics processing unit, hardware accelerator, field programmable gate array, neural network processing unit, artificial intelligence processing unit, inference engine, data processing unit, or infrastructure processing unit.

FIGS. 10A-10B depict another example computing device 1000 which may have any of the characteristics of computing device 900. FIG. 10A depicts the assembled computing device 1000 while FIG. 10B depicts an exploded view of the computing device 1000.

As FIG. 10B shows, the housing of computing device 1000 may comprise a bottom panel 1002, a rear panel 1004 with fins for airflow and apertures for power and video cabling, a front panel 1006 with apertures for light sources and/or camera lenses of motion capture devices 114A and 114B, and a top panel 1008.

Referring again to FIG. 1, computing device 118 may include any one or more electronic computing devices operable to receive, transmit, process, and store any appropriate data. In various embodiments, computing device 118 may include a mobile device or a stationary device capable of connecting (e.g., wirelessly) to one or more networks 110, motion capture devices 114, or displays 116. As examples, mobile devices may include laptop computers, tablet computers, smartphones, and other devices while stationary devices may include desktop computers, televisions (e.g., computing device 118 may be integrated with display 116), or other devices that are not easily portable. Computing device 118 may include a set of programs such as operating systems (e.g., Microsoft Windows, Linux, Android, Mac OSX, Apple iOS, UNIX, or other operating system), applications, plug-ins, applets, virtual machines, machine images, drivers, executable files, and other software-based programs capable of being run, executed, or otherwise used by computing device 118.

Backend system 104 may comprise any suitable servers or other computing devices that facilitate the provision of features of the system 100 as described herein. In various embodiments, backend system 104 or any components thereof may be deployed using a cloud service such as Amazon Web Services, Microsoft Azure, or Google Cloud Platform. For example, the functionality of the backend system 104 may be provided by virtual machine servers that are deployed for the purpose of providing such functionality or may be provided by a service that runs on an existing platform. In one embodiment backend system 104 may include a backend server that communicates with a database to initiate storage and retrieval of data related to the system 100. The database may store any suitable data associated with the system 100 in any suitable format(s). For example, the database may include one or more database management systems (DBMS), such as SQL Server, Oracle, Sybase, IBM DB2, or NoSQL databases (e.g., Redis and MongoDB).

Application server 106 may be coupled to one or more computing devices through one or more networks 110. One or more applications that may be used in conjunction with system 100 may be supported with, downloaded from, served by, or otherwise provided through application server 106 or other suitable means. In some instances, the applications can be downloaded from an application storefront onto a particular computing device using storefronts such as Google Android Market, Apple App Store, Palm Software Store and App Catalog, RIM App World, etc., or other sources. As an example, a user 112 may use an application to provide information about physical attributes, fitness goals, or other information to the system 100 and use the application to receive feedback from the system 100 (e.g., workout information or other suitable information). As another example, experts in the expert network system 108 may use an application to receive information about a user 112 and provide recommended workout information to the system 100.

In general, servers and other computing devices of backend system 104 or application server 106 may include electronic computing devices operable to receive, transmit, process, store, or manage data and information associated with system 100. As used in this document, the term computing device, is intended to encompass any suitable processing device. For example, portions of backend system 104 or application server 106 may be implemented using servers (including server pools) or other computers. Further, any, all, or some of the computing devices may be adapted to execute any operating system, including Linux, UNIX, Windows Server, etc., as well as virtual machines adapted to virtualize execution of a particular operating system, including customized and proprietary operating systems.

In some embodiments, multiple backend systems 104 may be utilized. For example, a first backend system 104 may be used to support the operations of system 102 and a second backend system 104 may be used to support the operations of system 120.

Servers and other computing devices of system 100 can each include one or more processors, computer-readable memory, and one or more interfaces, among other features and hardware. Servers can include any suitable software component or module, or computing device(s) capable of hosting and/or serving a software application or services (e.g., services of backend system 104 or application server 106), including distributed, enterprise, or cloud-based software applications, data, and services. For instance, servers can be configured to host, serve, or otherwise manage data sets, or applications interfacing, coordinating with, or dependent on or used by other services. In some instances, a server, system, subsystem, or computing device can be implemented as some combination of devices that can be hosted on a common computing device, server, server pool, or cloud computing environment and share computing resources, including shared memory, processors, and interfaces.

Computing devices used in system 100 (e.g., computing devices 118 or computing devices of expert network system 108 or backend system 104) may each include a computer system to facilitate performance of their respective operations. In particular embodiments, a computer system may include a processor, memory, and one or more communication interfaces, among other components. These components may work together in order to provide functionality described herein.

A processor may be a microprocessor, controller, or any other suitable computing device, resource, or combination of hardware, stored software and/or encoded logic operable to provide, either alone or in conjunction with other components of computing devices, the functionality of these computing devices. For example, a processor may comprise a processor core, graphics processing unit, hardware accelerator, application specific integrated circuit (ASIC), field programmable gate array (FPGA), neural network processing unit, artificial intelligence processing unit, inference engine, data processing unit, or infrastructure processing unit. In particular embodiments, computing devices may utilize multiple processors to perform the functions described herein.

A processor can execute any type of instructions to achieve the operations detailed herein. In one example, the processor could transform an element or an article (e.g., data) from one state or thing to another state or thing. In another example, the activities outlined herein may be implemented with fixed logic or programmable logic (e.g., software/computer instructions executed by the processor) and the elements identified herein could be some type of a programmable processor, programmable digital logic (e.g., FPGA), an erasable programmable read only memory (EPROM), an electrically erasable programmable ROM (EEPROM)), or an ASIC that includes digital logic, software, code, electronic instructions, or any suitable combination thereof.

Memory may comprise any form of non-volatile or volatile memory including, without limitation, random access memory (RAM), read-only memory (ROM), magnetic media (e.g., one or more disk or tape drives), optical media, solid state memory (e.g., flash memory), removable media, or any other suitable local or remote memory component or components. Memory may store any suitable data or information utilized by computing devices, including software embedded in a computer readable medium, and/or encoded logic incorporated in hardware or otherwise stored (e.g., firmware). Memory may also store the results and/or intermediate results of the various calculations and determinations performed by processors.

Communication interfaces may be used for the communication of signaling and/or data between computing devices and one or more networks (e.g., 110) or network nodes or other devices of system 100. For example, communication interfaces may be used to send and receive network traffic such as data packets. Each communication interface may send and receive data and/or signals according to a distinct standard such as an IEEE 802.11, IEEE 802.3, or other suitable standard. In some instances, communication interfaces may include antennae and other hardware for transmitting and receiving radio signals to and from other devices in connection with a wireless communication session.

System 100 also includes network 110 to communicate data between the system 102, the backend system 104, the application server 106, and expert network system 108. Network 110 may be any suitable network or combination of one or more networks operating using one or more suitable networking protocols. A network may represent a series of points, nodes, or network elements and interconnected communication paths for receiving and transmitting packets of information. For example, a network may include one or more routers, switches, firewalls, security appliances, antivirus servers, or other useful network elements. A network may provide a communicative interface between sources and/or hosts, and may comprise any public or private network, such as a local area network (LAN), wireless local area network (WLAN), metropolitan area network (MAN), Intranet, Extranet, Internet, wide area network (WAN), virtual private network (VPN), cellular network (implementing GSM, CDMA, 3G, 4G, 5G, LTE, etc.), or any other appropriate architecture or system that facilitates communications in a network environment depending on the network topology. A network can comprise any number of hardware or software elements coupled to (and in communication with) each other through a communications medium. In some embodiments, a network may simply comprise a transmission medium such as a cable (e.g., an Ethernet cable), air, or other transmission medium.

“Logic” as used herein, may include but not be limited to hardware, firmware, software and/or combinations of each to perform a function(s) or an action(s), and/or to cause a function or action from another logic, method, and/or system. In various embodiments, logic may include a software controlled microprocessor, discrete logic (e.g., an application specific integrated circuit (ASIC)), a programmed logic device (e.g., a field programmable gate array (FPGA)), a memory device containing instructions, combinations of logic devices, or the like. Logic may include one or more gates, combinations of gates, or other circuit components. Logic may also be fully embodied as software.

The functionality described herein may be performed by any suitable component(s) of the system. For example, certain functionality described herein as being performed by system 102 may be performed by backend system 104 or by a combination of system 102 and backend system 104. Similarly, certain functionality described herein as being performed by computing device 118 may be performed by backend system 104 or by a combination of computing device 118 and backend system 104.

While the present disclosure has been described with respect to a limited number of embodiments, those skilled in the art will appreciate numerous modifications and variations therefrom. It is intended that the appended claims cover all such modifications and variations as fall within the true spirit and scope of this present disclosure. Although this disclosure has been described in terms of certain implementations and generally associated methods, alterations and permutations of these implementations and methods will be apparent to those skilled in the art. For example, the actions described herein can be performed in a different order than as described and still achieve the desirable results. As one example, the processes depicted in the accompanying figures do not necessarily require the particular order shown, or sequential order, to achieve the desired results. In certain implementations, multitasking and parallel processing may be advantageous. Other variations are within the scope of the following claims.

The architectures presented herein are provided by way of example only, and are intended to be non-exclusive and non-limiting. Furthermore, the various parts disclosed are intended to be logical divisions only, and need not necessarily represent physically separate hardware and/or software components. Certain computing systems may provide memory elements in a single physical memory device, and in other cases, memory elements may be functionally distributed across many physical devices. In the case of virtual machine managers or hypervisors, all or part of a function may be provided in the form of software or firmware running over a virtualization layer to provide the disclosed logical function.

Note that with the examples provided herein, interaction may be described in terms of a single computing system. However, this has been done for purposes of clarity and example only. In certain cases, it may be easier to describe one or more of the functionalities of a given set of flows by only referencing a single computing system. Moreover, the system for deep learning and malware detection is readily scalable and can be implemented across a large number of components (e.g., multiple computing systems), as well as more complicated/sophisticated arrangements and configurations. Accordingly, the examples provided should not limit the scope or inhibit the broad teachings of the computing system as potentially applied to a myriad of other architectures.

As used herein, unless expressly stated to the contrary, use of the phrase ‘at least one of’ refers to any combination of the named items, elements, conditions, or activities. For example, ‘at least one of X, Y, and Z’ is intended to mean any of the following: 1) at least one X, but not Y and not Z; 2) at least one Y, but not X and not Z; 3) at least one Z, but not X and not Y; 4) at least one X and at least one Y, but not Z; 5) at least one X and at least one Z, but not Y; 6) at least one Y and at least one Z, but not X; or 7) at least one X, at least one Y, and at least one Z.

Additionally, unless expressly stated to the contrary, the terms ‘first’, ‘second’, ‘third’, etc., are intended to distinguish the particular nouns (e.g., element, condition, module, activity, operation, claim element, etc.) they modify, but are not intended to indicate any type of order, rank, importance, temporal sequence, or hierarchy of the modified noun. For example, ‘first X’ and ‘second X’ are intended to designate two separate X elements that are not necessarily limited by any order, rank, importance, temporal sequence, or hierarchy of the two elements.

References in the specification to “one embodiment,” “an embodiment,” “some embodiments,” etc., indicate that the embodiment(s) described may include a particular feature, structure, or characteristic, but every embodiment may or may not necessarily include that particular feature, structure, or characteristic. Moreover, such phrases are not necessarily referring to the same embodiment.

While this specification contains many specific implementation details, these should not be construed as limitations on the scope of any embodiments or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, the separation of various system components and modules in the embodiments described above should not be understood as requiring such separation in all embodiments. It should be understood that the described program components, modules, and systems can generally be integrated together in a single software product or packaged into multiple software products.

Use of the phrase ‘configured to,’ in one embodiment, refers to arranging, putting together, manufacturing, offering to sell, importing and/or designing an apparatus, hardware, logic, or element to perform a designated or determined task. In this example, an apparatus or element thereof that is not operating is still ‘configured to’ perform a designated task if it is designed, coupled, and/or interconnected to perform said designated task. Note once again that use of the term ‘configured to’ does not require operation, but instead focus on the latent state of an apparatus, hardware, and/or element, where in the latent state the apparatus, hardware, and/or element is designed to perform a particular task when the apparatus, hardware, and/or element is operating.

Furthermore, use of the phrases ‘to,’ ‘capable of/to,’ and or ‘operable to,’ in one embodiment, refers to some apparatus, logic, hardware, and/or element designed in such a way to enable use of the apparatus, logic, hardware, and/or element in a specified manner. Note as above that use of to, capable to, or operable to, in one embodiment, refers to the latent state of an apparatus, logic, hardware, and/or element, where the apparatus, logic, hardware, and/or element is not operating but is designed in such a manner to enable use of an apparatus in a specified manner.

The embodiments of methods, hardware, software, firmware or code set forth above may be implemented via instructions or code stored on a machine-accessible, machine readable, computer accessible, or computer readable medium which are executable by a processing element. A machine-accessible/readable medium includes any mechanism that provides (e.g., stores and/or transmits) information in a form readable by a machine, such as a computer or electronic system. For example, a non-transitory machine-accessible medium includes random-access memory (RAM), such as static RAM (SRAM) or dynamic RAM (DRAM); ROM; magnetic or optical storage medium; flash memory devices; electrical storage devices; optical storage devices; acoustical storage devices; other form of storage devices for holding information received from transitory (propagated) signals (e.g., carrier waves, infrared signals, digital signals); etc., which are to be distinguished from the non-transitory mediums that may receive information there from.

Instructions used to program logic to perform embodiments of the invention may be stored within a memory in the system, such as DRAM, cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, Compact Disc, Read-Only Memory (CD-ROMs), and magneto-optical disks, Read-Only Memory (ROMs), Random Access Memory (RAM), Erasable Programmable Read-Only Memory (EPROM), Electrically Erasable Programmable Read-Only Memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the computer-readable medium includes any type of tangible machine-readable medium suitable for storing or transmitting electronic instructions or information in a form readable by a machine (e.g., a computer).

Numerous other changes, substitutions, variations, alterations, and modifications may be ascertained to one skilled in the art and it is intended that the present disclosure encompass all such changes, substitutions, variations, alterations, and modifications as falling within the scope of the appended claims.

1. A method, comprising:
generating, for display to a user, a representation of the user performing a movement pattern of an activity from a first point of view;
sensing a deviation of movement of the user from a model movement pattern for the activity;
selecting a second point of view based on a type of the deviation; and
generating, for display to the user, a representation of the user performing the movement pattern for the activity from the second point of view.

2. The method of claim 1, further comprising:
providing, for display to the user, a representation of a trainer performing an example movement pattern of the activity from the first point of view; and
providing, for display to the user, a representation of the trainer performing the example movement pattern of the activity from the second point of view responsive to sensing the deviation of the user from the model movement pattern.

3. The method of claim 1, further comprising generating a guide skeleton to be overlaid on the representation of the user.

4. The method of claim 3, further comprising changing a color or brightness of a segment of the guide skeleton responsive to alignment of a body segment of the user with the segment of the guide skeleton.

5. The method of claim 3, further comprising causing display of the guide skeleton responsive to a determination that the user is near a target position associated with the deviation.

6. The method of claim 1, wherein the representation of the user performing the movement pattern from the first point of view and the representation of the user performing the movement pattern from the second point of view is based on video data captured by image sensors on separate motion capture devices while the user maintains the same orientation with respect to the image sensors.

7. The method of claim 1, further comprising generating a score indicative of a similarity between the movement of the user and the model movement pattern for display to the user.

8. At least one non-transitory computer-readable storage medium including machine-readable instructions which, when executed, cause a machine to:
generate, for display to a user, a representation of the user performing a movement pattern of an activity from a first point of view;
sense a deviation of movement of the user from a model movement pattern for the activity;
select a second point of view based on a type of the deviation; and
generate, for display to the user, a representation of the user performing the movement pattern for the activity from the second point of view.

9. The medium of claim 8, the instructions, when executed, to cause the machine to:
provide, for display to the user, a representation of a trainer performing an example movement pattern of the activity from the first point of view; and
provide, for display to the user, a representation of the trainer performing the example movement pattern of the activity from the second point of view responsive to sensing the deviation of the user from the model movement pattern.

10. The medium of claim 8, the instructions, when executed, to cause the machine to generate a guide skeleton to be overlaid on the representation of the user.

11. The medium of claim 10, the instructions, when executed, to cause the machine to change a color or brightness of a segment of the guide skeleton responsive to alignment of a body segment of the user with the segment of the guide skeleton.

12. The medium of claim 10, the instructions, when executed, to cause the machine to cause display of the guide skeleton responsive to a determination that the user is near a target position associated with the deviation.

13. The medium of claim 8, wherein the representation of the user performing the movement pattern from the first point of view and the representation of the user performing the movement pattern from the second point of view is based on video data captured by image sensors on separate motion capture devices while the user maintains the same orientation with respect to the image sensors.

14. The medium of claim 8, the instructions, when executed, to cause the machine to generate a score indicative of a similarity between the movement of the user and the model movement pattern for display to the user.

15. A system comprising:
a computing system comprising:
a memory to store information indicative of a model movement pattern for an activity; and
at least one processor to:
generate, for display to a user, a representation of the user performing a movement pattern of the activity from a first point of view;
sense a deviation of movement of the user from the model movement pattern for the activity;
select a second point of view based on a type of the deviation; and
generate, for display to the user, a representation of the user performing the movement pattern for the activity from the second point of view.

16. The system of claim 15, further comprising a plurality of motion capture devices to capture positional data representing the movement of the user.

17. The system of claim 16, wherein the representation of the user performing the movement pattern from the first point of view and the representation of the user performing the movement pattern from the second point of view is based on video data captured by image sensors on the motion capture devices while the user maintains the same orientation with respect to the image sensors.

18. The system of claim 15, further comprising a display coupled to the computing system, the display to display the representation of the user from the first and second points of view.

19. The system of claim 15, wherein the computing system is to:
provide, for display to the user, a representation of a trainer performing an example movement pattern of the activity from the first point of view; and
provide, for display to the user, a representation of the trainer performing the model movement pattern of the activity from the second point of view responsive to sensing the deviation of the user from the model movement pattern.

20. The system of claim 15, wherein the computing system is to generate a guide skeleton to be overlaid on the representation of the user.
